(19) United States
(12) Patent Application Publication
(10) Pub. No.: US 2004/0064113 A1
(43) Pub. Date: Apr. 1, 2004

(54) Disposable absorbent article with wetness/dryness indicator

(76) Inventor: Carol L. Erdman, West Chester, PA (US)

Correspondence Address: HUNTON & WILLIAMS LLP, INTELLECTUAL PROPERTY DEPARTMENT, 1900 K STREET, N.W., SUITE 1200, WASHINGTON, DC, 20006-1109, US

(21) Appl. No.: 10/255,108

(22) Filed: Sep. 26, 2002

(51) Int. Cl. A61F 13/15; A61F 13/20

(52) U.S. Cl. 604/361

(57) ABSTRACT

This invention relates to the field of absorbent articles in general, and more particularly to absorbent articles that have wetness/dryness indicators disposed thereon. Preferably, the disposable absorbent article, for example, a training pant, includes a disappearing wetness/dryness indicator that disappears when the article is wetted, and an appearing wetness/dryness indicator that appears when the article is wetted. The wetness/dryness indicators preferably allow a caregiver to interact with a child to promote toilet training. The wetness/dryness indicators are graphic elements that may or may not be related to one another, and that may or may not be related to other graphic elements on the article.

### BACKGROUND OF THE INVENTION

1. Field of the Invention

This invention relates to the field of absorbent articles in general, and more particularly to absorbent articles that have wetness/dryness indicators disposed thereon. Preferably, the disposable absorbent article, for example, a training pant, includes wetness/dryness indicators that are present when the article is dry, and that disappear when the article is wetted, and includes additional wetness/dryness indicators that are not present when the article is dry, and that appear when the article is wetted. The wetness/dryness indicators preferably allow a caregiver to interact with a child to promote toilet training.

2. Description of Related Art

Disposable absorbent articles typically include a moisture-impervious backing sheet, an absorbent pad, and a moisture permeable liner sheet (e.g., top sheet) that contacts the body of a person wearing the article. In addition, elasticized regions are provided around the edges of the article to secure the article about the waist and legs of a wearer. Diapers typically further comprise opposed front and rear waist portions defining a waist opening, a crotch portion disposed there between, and a pair of elastically contractible leg openings along the side edges of the crotch portion. Disposable diapers having elasticized margins for placement about the legs of a wearer are disclosed in U.S. Pat. No. 4,050,462 and U.S. Pat. No. 5,092,861. An absorbent article having elasticized side margins and waist band margins are shown in U.S. Pat. No. 4,300,562. These elasticized portions of the garment typically are designed to contain body exudates and hence, prevent leakage.

Training pants today are being designed to include a number of different types of graphics in an attempt to train the child to go to the bathroom on the toilet, and to persuade a child to wear conventional undergarments. For example, some of these graphics include markings that make the training pants look like conventional boys or girls undergarments with the seams highlighted or emphasized to make the garment appear more like a conventional brief (e.g., simulated fly openings for boys, ruffles for girls, etc.).

Potty training a young child typically includes a wide variety of different aspects, including many training techniques and training aids that may be used by parents and caregivers, hereinafter simply referred to as caregivers. One feature of potty or toilet training is having the young child change from wearing diapers to wearing training pants to help the child understand that he or she should now use the toilet just like adults. An additional feature of the potty training process includes caregiver instruction as a positive encouragement and reinforcement to the child that he or she should now be using a toilet instead of diapers. Although the use of training pants and positive encouragement from the caregiver has been helpful in the toilet training process, there is still much room for improvement. Specifically, caregivers continue to search for easier and quicker ways to guide their children successfully through the potty training process.

Many caregivers and parents have difficulty in determining when the child is ready to begin potty training, and underestimate the difficulty of teaching young children to use the toilet like an adult. Parents attempt many different forms of positive enforcement, from potty stickers or stamps, to using floating objects in the toilet for little boys to take aim. If a child does not respond to an initial toilet training instruction or introduction, the caregiver may be at a loss for finding techniques, methods, or teaching tools to encourage the child to use the toilet. Thus, while various teaching tools such as books, videotapes, charts with stickers, personalized toilets, and interactive toilet training kits are available, there remains a need for new and improved educational and motivational mechanisms to facilitate the toilet training process.

Another problem facing caregivers is that they typically do not know when a child has had an accident in his or her training pants, which can help in the potty training process since the accident will still be fresh in the child's mind. Wetness indicators have been used as a mechanism to assist caregivers in knowing when the articles has been wetted. The art is rife with disclosures on types of wetness indicators, whether they be disappearing inks, appearing inks, inks activated by heat and the like, etc. Typical wetness indicators are disclosed, for example, in U.S. Pat. Nos. 4,022,211, 4,292,916, 4,812,053, 4,903,254, 4,987,849, 5,045,283, 5,503,339, 5,058,088, 6,297,424, and 6,307,119, the disclosures of each of which is incorporated by reference herein in their entirety.

U.S. Pat. Nos. 6,297,424 and 6,307,119 disclose the use of various wetness indicators (called “active object graphics”) and their relationship with other graphics on the training patent (e.g., being “interactively interrelated” or “unrelated in subject matter”). The relationship between the various graphics, as well as their positioning, or registration, on the absorbent article, is said to assist the caregiver in telling a story to the child to assist in the potty training process. The use of these complicated interrelationships and registered positioning adds significantly to the cost of training pants, however, making them an undesirable purchase for most caregivers.

The description herein of the various known products, methods, and apparatus and their attendant disadvantages is in no way intended to limit the scope of the present invention, or to imply that the present invention does not include some or all of the various elements of the products, methods, and apparatus in one form or another. Indeed, various embodiments of the invention may be capable of overcoming some of the disadvantages noted herein, while still retaining some or all of the various elements of the known products, methods, and apparatus in one form or another.

All documents described herein are incorporated by reference in this disclosure in their entirety.

### SUMMARY OF THE INVENTION

It therefore is desirable to provide cost-efficient absorbent articles that assist in the potty training process. It also is desirable to provide absorbent articles that help a caregiver teach a young child to use the toilet using positive reinforcement.

In accordance with features of various embodiments of the present invention, there is provided an absorbent article that includes at least one disappearing graphic and at least one appearing graphic that disappear and appear respectively, when the absorbent article is insulted with an average insult amount of urine. In accordance with an additional feature of an embodiment of the invention, there is provided an absorbent article that includes a top sheet, a back sheet, and an absorbent core disposed at least partially between the top sheet and the back sheet. Disposed on the absorbent core facing surface of the back sheet are at least one disappearing graphic and at least one appearing graphic that disappear and appear respectively, when the absorbent article is insulted with an average insult amount of urine.

In accordance with an additional feature of an embodiment of the invention, there is provided a method of making an absorbent article that includes providing a top sheet, a back sheet, and an absorbent core to a garment forming station. The method includes disposing the absorbent core at least partially between the top sheet and the back sheet, and then disposing at least one disappearing graphic and at least one appearing graphic at least partially between the back sheet and the absorbent core. In accordance with preferred embodiments of the method, the graphics can be imprinted at the same or different times, and they can be either printed directly on a surface of the back sheet, or on an additional layer(s) and then disposed between the absorbent core and the back sheet.

The absorbent article may include any number of layers in addition to the back sheet, top sheet and absorbent core, and the disappearing graphics, and appearing graphics may be imprinted or otherwise disposed on the same or different ones of these layers. The absorbent article also may include additional graphics that may or may not be related to the disappearing or appearing graphics.

These and other features of various embodiments of the invention will be readily apparent from a review of the detailed description of preferred embodiments taken in conjunction with the attached drawings.

### BRIEF DESCRIPTION OF THE DRAWINGS

FIG. 1 is a front plan view of a pants-type absorbent garment with portions of the back sheet removed to reveal inner components of the garment;

FIG. 2 is a schematic of the various parts of a pants-type absorbent garment;

FIGS. 3a and 3b are front views of a preferred embodiment of the invention showing the transformation from a disappearing graphic to an appearing graphic;

FIGS. 4a and 4b are front views of a preferred embodiment of the invention showing the transformation from a disappearing graphic to a appearing graphic;

FIGS. 5a and 5b are front views of a preferred embodiment of the invention showing the transformation from a disappearing graphic to an appearing graphic;

FIGS. 6a and 6b are front views of a preferred embodiment of the invention showing the transformation from a disappearing graphic to an appearing graphic; and

FIG. 7 is an illustration of an apparatus and method useful for making an absorbent garment of the invention.

### DETAILED DESCRIPTION OF PREFERRED EMBODIMENTS

As used herein, the terms “absorbent garment,” “absorbent article” or simply “article” or “garment” refer to devices that absorb and contain body fluids and other body exudates. More specifically, these terms refer to garments that are placed against or in proximity to the body of a wearer to absorb and contain the various exudates discharged from the body. A non-exhaustive list of examples of absorbent garments includes diapers, diaper covers, disposable diapers, training pants, feminine hygiene products and adult incontinence products. Such garments may be intended to be discarded or partially discarded after a single use (“disposable” garments). Such garments may comprise essentially a single inseparable structure (“unitary” garments), or they may comprise replaceable inserts or other interchangeable parts.

Absorbent garments and diapers may have a number of different constructions. In each of these constructions it is generally the case that an absorbent core is at least partially disposed between a liquid pervious, body-facing top sheet, and a liquid impervious, exterior back sheet. In some cases, one or both of the top sheet and back sheet may be shaped to form a pants-like garment. In other cases, the top sheet, back sheet and absorbent core may be formed as a discrete assembly that is placed on a main chassis layer and the chassis layer is shaped to form a pants-like garment. The garment may be provided to the consumer in the fully assembled pants-like shape, or may be partially pants-like and require the consumer to take the final steps necessary to form the final pant-like shape. In the case of training pants-type garments and most adult incontinent products, the garment is provided fully formed with factory-made side seams and the garment is donned by pulling it up the wearer's legs. In the case of diapers, a caregiver usually wraps the diaper around the wearer's waist and joins the side seams manually by attaching one or more adhesive or mechanical tabs, thereby forming a pant-like structure.

For clarity, the present invention is described herein only with reference to a pants-type garment in which the top sheet, back sheet and absorbent core are assembled onto a chassis layer that forms a pants-like garment, although the invention may be used with other constructions, and it is readily apparent and understood that this is not intended to limit the invention. The present invention may be used with any other absorbent garment that can be used as a teaching aid to teach a wearer (e.g., young child, accident victim, elderly person, etc.) to use the toilet.

The term “component” can refer, but is not limited to designated selected regions, such as edges, corners, sides or the like; structural members, such as elastic strips, absorbent pads, stretchable layers or panels, layers of material, or the like; or a graphic.

Throughout this description, the term “disposed” and the expressions “disposed on,” “disposing on,” “disposed in,” “disposed between” and variations thereof (e.g., a description of the article being “disposed” is interposed between the words “disposed” and “on”) are intended to mean that one element can be integral with another element, or that one element can be a separate structure bonded to or placed with or placed near another element. Thus, a component that is “disposed on” an element of the absorbent garment can be formed or applied directly or indirectly to a surface of the element, formed or applied between layers of a multiple layer element, formed or applied to a substrate that is placed with or near the element, formed or applied within a layer of the element or another substrate, or other variations or combinations thereof.

Throughout this description, the terms “top sheet” and “back sheet” denote the relationship of these materials or layers with respect to the absorbent composite core. It is understood that additional layers may be present between the absorbent composite core and the top sheet and back sheet, and that additional layers and other materials may be present on the side opposite the absorbent composite core from either the top sheet or the back sheet.

Throughout this description, the expression “disappearing graphic” denotes any graphical representation that disappears from view, as seen by the naked eye when insulted with a normal insult amount of urine. The expression “appearing graphic” denotes any graphical representation that appears from view, as seen by the naked eye when insulted with a normal insult amount of urine.

The term “positive” as it is used herein to refer to a positive disappearing graphic denotes any type of graphic that generally connotes a positive or sanguine image to a viewer. It is to be understood that not all viewers need to recognize the graphic as positive or sanguine, only that the graphic generally connote a sanguine image. While not intending to be limited to any specific examples of positive graphics, suitable positive graphics include smiling animals, people, anthropomorphic characters, sunny days, cartoons and cartoon characters, peaceful settings such as sunsets, oceans, lakes, waves, streams, living animals and other creatures, sporting events, flowers, gardens, angelic and other heavenly or religious images, and the like. Positive images also may include words that connote a positive spirit such as “dry,” “good boy,” “good girl,” “big boy”, “big girl,” “happy,” “smile” and other words and phrases.

The term “negative” as it is used herein to refer to a negative appearing graphic denotes any type of graphic that generally connotes a negative or disapproving or depressing image to a viewer. It is to be understood that not all viewers need to recognize the graphic as negative, disapproving, or depressing, only that the graphic generally connote a negative image. While not intending to be limited to any specific examples of negative graphics, suitable negative graphics include sad or crying animals, people, or anthropomorphic characters, gloomy days, cloudy days, unsettling settings such as dungeons, darkness, graveyards, mean cartoon characters, floods, fires, lightning, dead people, animals or other creatures, satanic characters, and the like. Negative graphics also may include words and phrases that connote a negative image, such as “oops,” “wet,” “bad boy,” “cry,” “sad,” and other words and phrases.

The invention includes one or more of each of the above-mentioned graphics arranged in such a manner that the disappearing graphics are visible on the absorbent garment when dry, and the appearing graphics are visible on the absorbent garment when wet, and in the area of wetness. It is preferred in the invention that the disappearing graphic be a positive disappearing graphic, and that the appearing graphic be a negative appearing graphic. It is preferred to include the graphics on two separate layers and dispose these layers on the absorbent core facing side or surface of the back sheet material. Alternatively, the graphics may be printed on top of one another on the same material. It also is possible in the invention to print one of the graphics, say the disappearing graphic, on the absorbent core facing surface of the back sheet, and printing the appearing graphic on the back sheet facing surface of the absorbent core (e.g. on the optional tissue layer which surrounds the absorbent material). In addition, the graphics may be printed on different sides of the same material.

Other embodiments and arrangements of the disappearing graphic and appearing graphic will be readily apparent to those skilled in the art upon reading this description, and consequently, are contemplated in the present invention. For example, the disappearing graphic and the appearing graphic may be disposed anywhere in the absorbent garment, and printed on any portion thereof, so long as the graphics are visible through the back sheet of the garment. Alternatively, the disappearing graphic and the appearing graphic may be disposed such that they are visible through the inner lining, as viewed from the inside of the absorbent article.

The present invention relates generally to absorbent garments, and in particular to a pants-type absorbent garment that contains a top sheet, a back sheet, and an absorbent core at least partially disposed between the top sheet and the back sheet. The absorbent garment of the invention preferably has a front waist region, a rear waist region and a crotch region positioned between the front and rear waist regions. Those skilled in the art recognize that “front” and “rear” in the context of the invention denote for clarity purposes only the front and rear of a user, and that the absorbent garment could be reversed whereby the previously described “front” portion becomes the rear portion, and vice versa.

The garment also preferably has a longitudinal centerline extending from the first waist region, through the crotch region and to the second waist region, and a lateral centerline orthogonal to the longitudinal centerline. The disposable pants-type absorbent article also may have disposed on an outer surface of the back sheet a disposal fastening mechanism that is capable of maintaining the garment in a rolled up configuration after the garment is rolled up. The disposal fastening mechanism may be a tape, a hook portion of a hook and loop fastener, and may include visible indicia to provide information to the caregiver or wearer of the disposable pants-type absorbent garment, or provide a graphic, or provide indicia indicating the origin of the article.

The invention also encompasses a method of making a disposable pants-type absorbent garment that includes providing a top sheet material, a back sheet material, and an absorbent core to a garment forming station. The method also includes disposing the absorbent core at least partially between the top sheet material and the back sheet material at the garment forming station. The method further includes providing a disappearing graphic and an appearing graphic, and disposing each graphic in such a manner that each is visible through the back sheet. It is preferred in the invention that the disappearing graphic be a positive disappearing graphic, and that the appearing graphic be a negative appearing graphic.

In the disposable pants-type absorbent garment of the invention, leg elastics preferably are provided along the leg openings for securely holding the leg openings against the thighs of the wearer to improve containment and fit. A pair of stand-up leg gathers or waist containment flaps may be attached to or formed from the body's side surface of the top sheet. Other elastic elements may be disposed in or on the absorbent garment to provide a firmer fit around the tummy and/or waist of the wearer.

The invention now will be described with reference to the attached drawings illustrating preferred embodiments of the invention. For clarity, features that appear in more than one Figure have the same reference number in each Figure. The following description refers to particularly preferred embodiments of the invention, such as the use of positive disappearing graphics and negative appearing graphics. Those skilled in the art will appreciate that the invention is not limited to these graphics and that any disappearing and appearing graphics can be used.

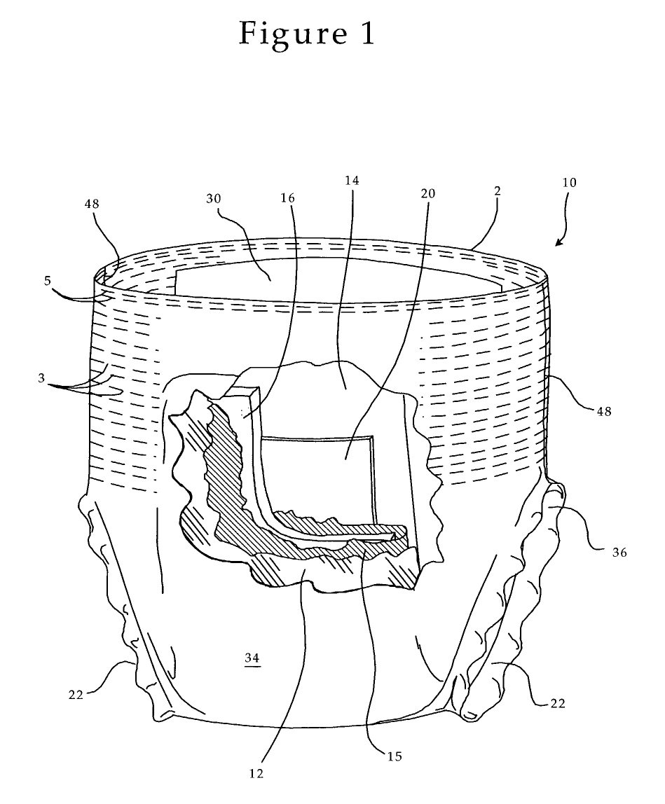

FIG. 1 depicts an embodiment of the present invention as it appears when worn by a user, with the main body partially cut away to show the absorbent core 16, and other internal components including, for example, an additional layer 20 (e.g., fluid transfer or handling layer, fluid acquisition layer, additional storage layer, wicking layer, and the like), and an optional tissue layer(s) 15 surrounding the core 16. In the depicted embodiment, the garment 10 is comprised of a main body 34 having an exterior facing liquid impervious outer layer 12 or “back sheet,” and a moisture pervious body-contacting inner layer 14 or “top sheet.” An absorbent core 16 is at least partially disposed between the top sheet 14 and the back sheet 12. In the embodiment depicted in FIG. 1 the back sheet 12, top sheet 14, and core 16 comprise the main body 34 of the garment, however in another embodiment of the invention the main body may be made from a separate sheet (for example, a garment chassis) and the back sheet 12, top sheet 14, and core 16 may be assembled separately then attached to the main body.

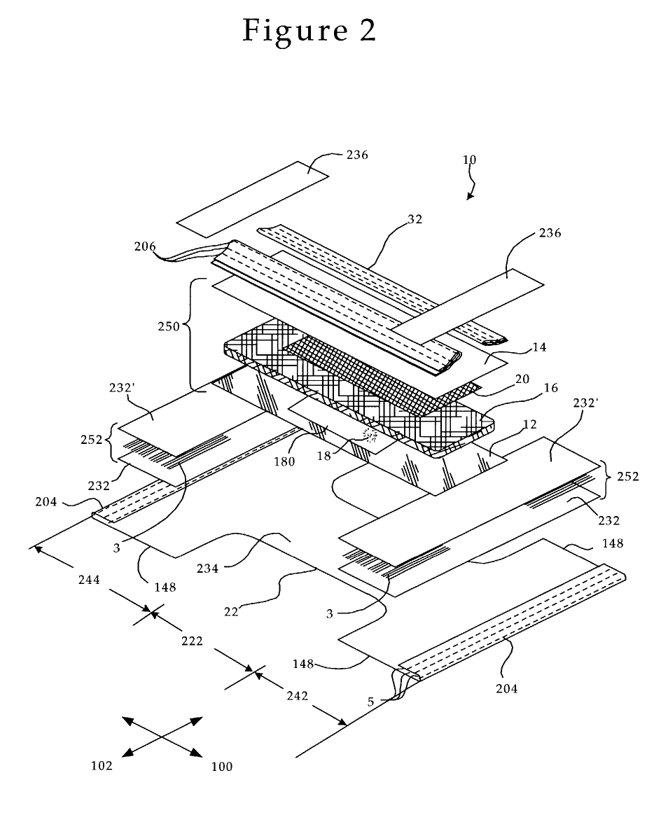

In the embodiment of the present invention depicted in FIG. 1, the garment 10 preferably further comprises various mechanisms for improving the fit of the garment 10 such as leg gathers 36 and standing leg gathers 32 (see, FIG. 2). Such gathers can be used to contract the leg holes 22 around the wearer's legs and body to prevent leakage. A garment 10 of the present invention may also comprise elastic or other fitting devices in the waist portions or other portions of the main body to help contain body exudates.

The various parts of the garment 10 are operatively associated with one another in such a manner that the garment will maintain its desired structure during use. The parts may be operatively associated with one another by a variety of methods known in the art, including, but not limited to: using adhesives such as hot melt adhesives and construction adhesives, chemical or solvent bonding, ultrasonic welding, stitching, heat bonding, or any other method of affixation known or hereafter discovered. All of the parts may be joined to each adjacent part, but some parts may not be joined to others. In one embodiment, the top sheet 14 and back sheet 12 are bonded to one another around their perimeter regions, thereby at least partially encasing and holding the absorbent core 16 in place without having to directly join the absorbent core 16 to any parts of the garment 10. The top sheet 14 or back sheet 12 also may be operatively associated with the absorbent core 16. As understood herein, the term “operatively associated” includes directly joining one part to another, indirectly joining parts together through one or more intermediary parts, whether those intermediary parts are described herein or not, joining parts in such a manner that unjoined parts are captured or held in their proper place, and any other suitable joining mechanism that maintains the structural integrity of the garment 10 for the duration of its use.

FIG. 2 is an exploded view of an embodiment of the present invention with elastic members shown in the elongated position for clarity, and the garment laid flat. The garment 10 has a longitudinal axis 100 corresponding approximately to the rear-to-front axis of the wearer, and a lateral axis 102, orthogonal to the longitudinal axis 100, and corresponding approximately to the side-to-side axis of the wearer. In one embodiment of the invention the lateral axis 102 of the garment 10 is approximately parallel with the machine direction of the garment 10.

In the embodiment of the invention depicted in FIG. 2, the main body 250 of the garment comprises a back sheet 12 and top sheet 14 having substantially identical dimensions. Those skilled in the art will recognize, however, that back sheet 12 and top sheet 14 need not have substantially identical dimensions; rather, either material may be smaller or larger than the other. The main body 250 of the absorbent garment 10 preferably is covered by an outer cover, more preferably, a chassis layer or layers 234. The perimeter of the chassis layer or layers is defined by laterally extending front and rear waist edges 204, longitudinally extending left and right front side edges 148 and left and right rear side edges 148, and leg holes 22. The laterally extending front waist edge 204, left and right front side edges 148, and at least part of leg holes 22 form a front waist region 242. The laterally extending rear waist edge 204, left and right rear side edges 148, and at least part of leg holes 22 form a rear waist region 244. The remaining portions of leg holes 22 form the crotch region 222.

Throughout this description, the terms “front,” “rear,” “left,” and “right” merely denote location relationships for purposes of explanation and clarity, and they generally relate to the location depicted in the drawings. Those skilled in the art appreciate that the front and rear of the absorbent garment may be reversed, as well as the left and right sides of the absorbent garment 10 depending upon the vantage point of the viewer.

To form the absorbent garment shown in FIG. 2 into a pants-type absorbent garment of the type shown in FIG. 1, the lateral edge portions 148 may be joined during or after manufacture by any mechanism known in the art or by a combination of such mechanisms. Examples of such mechanisms include: applying adhesives such as hot melt adhesives and construction adhesives, chemical or solvent bonding, stitching, heat bonding, autogenous bonding, and, preferably, ultrasonic welding. The lateral edge portions 148 also may be held proximal to one another or in an overlapping relationship during use by a fastener, such as a hook-and-loop fastener or adhesive fastener, as are well known in the art. When the lateral edge portions 148 are joined, leg hole cutouts along the lateral edges of the garment 10 form leg holes 22, and the longitudinal ends 104 of the garment 110 form a waist encircling edge 2 (FIG. 1). It is particularly preferred that lateral edge portions 148 be joined to one another via an elastically extensible material, or belt, that enables the sides of garment to expand. Alternatively, significant numbers of elastic materials 3 may be disposed near the lateral edges 148 to enable such expansion.

The top sheet 14 and back sheet 12 may be constructed from a wide variety of materials known in the art. The invention is not intended to be limited to any specific materials for these components. The top sheet 14 and back sheet 12 can be shaped and sized according to the requirements of each of the various types of absorbent garment, or to accommodate various user sizes. In an embodiment of the invention in which the garment 10 is a diaper or an adult incontinence brief, the combination of top sheet 14 and back sheet 12, may have an hourglass shape, or may have a rectangular shape, as seen in FIGS. 1 and 2, a trapezoidal “T” shape, or other shape.

Due to the wide variety of backing and liner sheet construction and materials currently available, the invention is not intended to be limited to any specific materials or constructions of these components. The back sheet 12 preferably is made from any suitable pliable liquid-impervious material known in the art. The selection and manufacture of such materials is well known in the art, and is disclosed, for example, in U.S. Pat. No. 6,123,694 issued to Peniak et al., and U.S. Pat. No. 6,176,952 issued to Maugans et al., the disclosure of each of which is incorporated herein by reference in its entirety. Typical back sheet materials include films of polyethylene, polypropylene, polyester, nylon, and polyvinyl chloride and blends of these materials. For example, the back sheet can be made of a polyethylene film having a thickness in the range of 0.02-0.04 mm. The back sheet 12 may be pigmented with, for example, titanium dioxide, to provide the garment 10 with a pleasing color or to render the back sheet 12 opaque enough that exudates being contained by the garment 10 are not visible from outside the garment, but yet transparent enough to allow graphic 18 to be visible. In addition, the back sheet 12 may be formed in such a manner that it is opaque, for example, by using various inert components in the polymeric film and then biaxially stretching the film. Other back sheet materials will be readily apparent to those skilled in the art. The back sheet 12 preferably has sufficient liquid imperviousness to prevent any leakage of fluids. The required level of liquid imperviousness may vary between different locations on the garment 10.

The back sheet 12 may further comprise separate regions having different properties. In a preferred embodiment, portions of the back sheet 12 are air-permeable to improve the breathability, and therefore comfort, of the garment 10. The different regions may be formed by making the back sheet 12 a composite of different sheet materials, chemical treatment, heat treatment, or other processes or methods known in the art. Some regions of the back sheet 12 may be fluid pervious. In one embodiment of the invention, the back sheet 12 is fluid impervious in the crotch region 222, but is fluid pervious in portions of the first and second waist regions 242, 244. The back sheet 12 may also be made from a laminate of overlaid sheets of material.

The moisture-pervious top sheet 14 can be comprised of any suitable relatively liquid-pervious material known in the art that permits passage of liquid there through. Non-woven liner sheet materials are exemplary because such materials readily allow the passage of liquids to the underlying absorbent core 16. Examples of suitable liner sheet materials include non-woven spun bond or carded webs of polypropylene, polyethylene, nylon, polyester and blends of these materials.

The back sheet 12 may be covered with a fibrous, non-woven fabric or chassis layer(s) 234 such as is disclosed, for example, in U.S. Pat. No. 4,646,362 issued to Heran et al., the disclosure of which is hereby incorporated by reference in its entirety and in a manner consistent with this disclosure. Materials for such a fibrous outer liner include a spun-bonded non-woven web of synthetic fibers such as polypropylene, polyethylene or polyester fibers; a non-woven web of cellulosic fibers, textile fibers such as rayon fibers, cotton and the like, or a blend of cellulosic and textile fibers; a spun-bonded non-woven web of synthetic fibers such as polypropylene; polyethylene or polyester fibers mixed with cellulosic, pulp fibers, or textile fibers; or melt blown thermoplastic fibers, such as macro fibers or micro fibers of polypropylene, polyethylene, polyester or other thermoplastic materials or mixtures of such thermoplastic macro fibers or micro fibers with cellulosic, pulp or textile fibers.

The chassis layer 234 also may comprise a non-woven polyethylene or polypropylene sheet, a polyethylene film, or any other suitable garment material known in the art or hereafter discovered. All or part of the chassis layer 234 may comprise a liquid pervious or liquid impervious material or a may be zone-treated to be partially liquid pervious or impervious. The chassis layer 234 may be stretched in one or more directions during the manufacturing process, thereby reducing its elasticity in the direction of stretch.

Alternatively, the back sheet 12 may comprise three panels wherein a central poly back sheet panel is positioned closest to absorbent core 16 while outboard non-woven breathable side back sheet panels are attached to the side edges of the central poly back sheet panel. Alternatively, the back sheet 12 may be formed from microporous poly coverstock for added breathability.

Although not illustrated in the drawings, the top sheet 14 may be formed of three separate portions or panels. Such an embodiment is disclosed, for example, in U.S. Pat. No. 5,275,590 issued to Huffman et al., which is incorporated herein by reference in its entirety, and in a manner consistent with the present invention. Those skilled in the art will recognize, however, that top sheet 14 need not be made of three separate panels, and that it may be comprised of one unitary item, or of a top sheet material 14, with separate standing leg gathers material 32 attached thereto, as shown in FIG. 2. A first top sheet panel may comprise a central top sheet panel formed from preferably a liquid-pervious material that is either hydrophobic or hydrophilic. The central top sheet panel may be made from any number of materials, including synthetic fibers (e.g., polypropylene or polyester fibers), natural fibers (e.g., wood or cellulose), apertured plastic films, reticulated foams and porous foams to name a few. One preferred material for a central top sheet panel is a cover stock of single ply non-woven material which may be made of carded fibers, either adhesively or thermally bonded, perforated plastic film, spunbonded fibers, or water entangled fibers, which generally weigh from 0.3-0.7 oz./sq. yd. and have appropriate and effective machine direction and cross-machine direction strength suitable for use as a baby diaper cover stock material. The central top sheet panel preferably extends from substantially the second waist region 244 to the first waist region 242, or a portion thereof.

The second and third top sheet panels (e.g., outer top sheet panels), in this alternative embodiment may be positioned laterally outside of the central top sheet panel. The outer top sheet panels preferably are substantially liquid-impervious and hydrophobic, preferably at least in the crotch area. The outer edges of the outer top sheet panels may substantially follow the corresponding outer perimeter of the back sheet 12. The material for the outer top sheet portions or panels preferably is polypropylene and can be woven, non-woven, spun bonded, carded or the like, depending on the application.

The respective top sheet panels may be attached to one another by, e.g., an adhesive. At the point of connection with the outer edges of the central top sheet panel and the inner edges of the outer top sheet panels extend upwardly to form waste containment flaps 32 (waist containment flaps 32, or standing leg gathers 32, are shown as separate elements in FIG. 2, but they need not be). The waste containment flaps 32 preferably are formed of the same material as the outer top sheet panels, as in the embodiment shown. They most preferably are an extension of the outer top sheet panels.

The waste containment flaps 32 may be treated with a suitable surfactant to modify their hydrophobicity/hydrophilicity as desired, and they may be treated with skin wellness ingredients to reduce skin irritation.

Alternatively, the waste containment flaps 32 may be formed as separate elements and then attached to the body side liner or top sheet 14. In this alternative embodiment, the central top sheet panel may extend past the connection point with the waste containment flaps 34, and even extend to the periphery of the back sheet 12.

The waste containment flaps 32 preferably include a portion that folds over onto itself to form a small enclosure. At least one, and depending on the size of the enclosure sometimes more than one, elastic element 206 may be secured in the enclosure in a stretched condition. As is well known in the art, when the flap elastic elements 206 attempt to assume the relaxed, unstretched condition, the waste containment flaps 32 rise above the surface of the center of the top sheet 14, as shown in FIG. 2.

The top sheet 14 may be made of any suitable relatively liquid-pervious material currently known in the art or later discovered that permits passage of a liquid there through. Examples of suitable top sheet materials include non-woven spun-bonded or carded webs of polypropylene, polyethylene, nylon, polyester and blends of these materials, perforated, apertured, or reticulated films, and the like. Non-woven materials are exemplary because such materials readily allow the passage of liquids to the underlying absorbent core 16. The top sheet 14 preferably comprises a single-ply non-woven material that may be made of carded fibers, either adhesively or thermally bonded, spun bonded fibers, or water entangled fibers, which generally weigh from 0.3-0.7 oz./sq. yd. and have appropriate and effective machine direction (longitudinal) and cross-machine (lateral) direction strength suitable for use as a top sheet material for the given application. The present invention is not intended to be limited to any particular material for the top sheet 14, and other top sheet materials will be readily apparent to those skilled in the art.

The top sheet 14 may further comprise several regions having different properties. In one embodiment of the present invention, the laterally distal portions of the top sheet 14, especially those used to make second and third top sheet panels, preferably are substantially fluid impervious and hydrophobic, while the remainder of the top sheet 14 (e.g., central top sheet panel) is hydrophilic and fluid pervious. Different top sheet properties, such as fluid perviousness and hydrophobicity, may be imparted upon the top sheet 14 by treating the top sheet 14 with adhesives, surfactants, or other chemicals, using a composite of different materials, or by other means. The top sheet 14 may also be made from a laminate of overlaid sheets of material. The top sheet 14 also may be treated in specific areas like the crotch region, with skin wellness ingredients such as aloe, vitamin E, and the like.

As noted elsewhere herein, the top sheet 14 and back sheet 12 may be substantially coterminous, or they may have different shapes and sizes. The particular design of the top sheet 14 and back sheet 12 may be dictated by manufacturing considerations, cost considerations, and performance considerations. Preferably, the top sheet 14 is large enough to completely cover the absorbent core 16, and the back sheet 12 is large enough to prevent leakage from the garment 10. The design of top sheet 14 and back sheet 12 is known in the art, and a skilled artisan will be able to produce an appropriate top sheet 14 and an appropriate back sheet 12 without undue experimentation.

The top sheet 14 and the back sheet 12 may be associated with one another using a variety of methods known in the art. For example, they may be thermally, ultrasonically, or chemically bonded to one another. They also may be joined using lines of hot melt adhesive or mechanical fasteners, such as thread, clips, or staples. In one embodiment, a hydrophilic adhesive, such as 34-5653 sold by National Starch, a corporation headquartered in Bridgewater, N.J., is used to join the top sheet 14 to the back sheet 12. The particular joining method may be dictated by the types of materials selected for the top sheet 14 and back sheet 12.

In one embodiment of the present invention, the top sheet 14 is operatively associated with the back sheet 12 around the perimeter of the top sheet 14. In this embodiment, the top sheet 14 and back sheet 12 may be operatively associated with one another by using hot melt adhesives, ultrasonic bonding, or any other suitable method known in the art. Also in this embodiment, the top sheet 14 and back sheet 12 may be bonded to one another in substantially all areas not having intermediately placed parts, such that some or all of the intermediately placed, or “sandwiched,” parts are physically captured between the top sheet 14 and back sheet 12, but not bonded to the back sheet 12 or top sheet 14.

An absorbent core 16 preferably is disposed between the inner surfaces of the back sheet 12 and the top sheet 14. The absorbent core 16 may be comprised of one or more layers of material, such as an absorbent layer for storing fluids and an acquisition layer for distributing fluids. Such multiple layer absorbent cores are known in the art and disclosed in U.S. Pat. No. 5,439,458 issued to Noel et al., which is incorporated herein by reference in its entirety, and in a manner consistent with the present invention.

The absorbent core 16 may be made from any absorbent material or materials known in the art. In one embodiment of the invention, the absorbent core 16 comprises wood fibers or other fibers such as tow fibers, chemical wood pulp, or any other suitable liquid absorbing material, such as commercially available fluff pulp or fluffed bleached kraft softwood pulp. In another embodiment of the invention, the absorbent core 16 comprises a combination of a porous fibrous web and super absorbent particles. Such absorbent cores are known in the art and are disclosed, for example, in U.S. Pat. No. 5,281,207 issued to Chmielewski et al., which is incorporated herein by reference in its entirety. In such an embodiment, the absorbent core 16 may be surrounded by a liquid pervious tissue over-wrap 15 (FIG. 1), or other material.

The absorbent core 16 also may be a laminate material, as described in U.S. Pat. No. 6,068,620, the disclosure of which is incorporated by reference herein in its entirety. Such laminate materials typically include outer tissue layers 15 (FIG. 1) surrounding a central fibrous layer 16 that contains a high percentage by weight of superabsorbent polymer (SAP). Any type of SAP can be used in this embodiment, or in any absorbent core 16 that is useful in this invention. The SAP generally is a water-insoluble but water-swellable polymeric substance capable of absorbing water in an amount which is at least ten times the weight of the substance in its dry form. In one type of superabsorbent material, the particles or fibers may be described chemically as having a back bone of natural or synthetic polymers with hydrophilic groups or polymers containing hydrophilic groups being chemically bonded to the back bone or in intimate admixture therewith. Included in this class of materials are such modified polymers as sodium neutralized cross-linked polyacrylates and polysaccharides including, for example, cellulose and starch and regenerated cellulose which are modified to be carboxylated, phosphonoalkylated, sulphoxylated or phosphorylated, causing the SAP to be highly hydrophilic. Such modified polymers may also be cross-linked to reduce their water-solubility.

Any SAP now known or later discovered can be used in the absorbent core 16 of the invention. Commercially available SAPs include a starch modified superabsorbent polymer available under the trade name SANWET® from BASF Corporation, Portsmouth, Va. SANWET® is a starch grafted polyacrylate sodium salt. Other commercially available SAPs include a superabsorbent derived from polypropenoic acid, available under the tradename DRYTECH® 520 SUPERABSORBENT POLYMER from The Dow Chemical Company, Midland Mich.; AQUA KEEP manufactured by Seitetsu Kagaku Co., Ltd.; ARASORB manufactured by Arakawa Chemical (U.S.A.) Inc.; ARIDALL 1125 manufactured by Chemdall Corporation; FAVOR manufactured by Stockhausen Inc.; HYSORB from BASF Atkienqesellshaft, Ludwigshafen, Germany; AQUA KEEP SA60S, manufactured by Seitetsu Kagaku Co., Ltd.; DIAWET, commercially available from Mitsubishi Chemicals, Japan; FLOSORB, available from SNF Floerger, France, AQUALIC, available from Nippon Shokubai, Osaka, Japan.

The SAP may be provided in any particle size, and suitable particle sizes vary greatly depending on the ultimate properties desired. For example, a fine particulate rather than a coarse particulate may be used in the invention, and preferably a fine particulate that passes through an about 200 mesh screen may be used.

The absorbent core 16 may be surrounded by a liquid pervious tissue over-wrap 15 (FIG. 1), or other material(s), which may be treated to be hydrophobic or hydrophilic, or to have other properties. The absorbent core 16, and any tissue wrap enclosing it, may be folded, crimped, thermally bonded, or otherwise manipulated to provide additional benefits. It is envisioned that a variety of folding patterns may be employed to provide additional fluid handling capabilities. For example, the absorbent core 16 may be folded into a U shape, a C shape, a G shape, a Z shape, or other shapes, as viewed along the longitudinal axis 100, to provide fluid handling channels, multiple layers of absorbent material, or other benefits. Folded absorbent cores are discussed, for example, in U.S. Pat. No. 6,068,620.

The absorbent core 16 generally is elongated along the longitudinal axis 100 of the garment, and may extend along either or both of the lateral axis 102 and the longitudinal axis 100 to the outer perimeter of the garment. In the embodiment depicted in FIGS. 1 and 2, the absorbent core 16 is substantially rectangular in shape, however, it also may have rounded ends or other shapes, such as an “I” shape or a “T” shape. The absorbent core 16 also may have channels, grooves or pockets, and may have a varying thickness. In an embodiment having a channeled or pocketed absorbent core 16, such channels or pockets may be substantially vacant, or may be filled with additional SAP or additional supplemental absorbent cores having similar or different properties than the absorbent core 16.

As mentioned previously, the various parts of the garment 10 preferably are operatively associated with one another in such a manner that the garment will maintain its desired structure during use. The parts may be operatively associated with one another by a variety of methods known in the art, including, but not limited to: using adhesives such as hot melt adhesives and construction adhesives, chemical or solvent bonding, ultrasonic welding, stitching, heat bonding, autogenous bonding, or any other method of affixation known or hereafter discovered. U.S. Pat. No. 4,919,738 issued to Ball et. al. discloses a method of autogenous bonding, and its disclosure is herein incorporated by reference in its entirety in a manner consistent with the invention. All of the parts may be joined to each adjacent part, but some parts may not be joined to others.

The core assembly 250 may comprise additional layers 20 of material that may reduce rewet of the top sheet 14, reduce strikethrough times or otherwise improve the absorbency, dryness and other properties of the garment 10. Examples of the one or more additional layers 20 include any layer selected from a fluid acquisition layer, a distribution layer, an additional fibrous layer optionally containing SAP, a wicking layer, a storage layer, a dryness layer, a softness layer, or combinations and fragments of these layers. Such layers may be provided to assist with transferring fluids to the absorbent core 16, handling fluid surges, preventing rewet, containing absorbent material, improving core stability, or for other purposes. Skilled artisans are familiar with the various additional layers 20 that may be included in an absorbent article, and the present invention is not intended on being limited to any particular type of materials used for those layers. Rather, the invention encompasses all types of wicking layers, all types of distribution layers, etc., to the extent that type of layer is utilized.

The core assembly 250 may be attached to the chassis layer 234 by any mechanism known in the art, such as by ultrasonic bonding or by the use of lines of hot melt adhesive. The bond between the core assembly 250 and the chassis layer 234 may be reinforced by laterally-extending end strips 236 that preferably are applied over the longitudinal ends of the core assembly 250 and bonded to the underlying structure of the garment 10. The end strips 236 also may hold the ends of the standing leg gathers 32 so that the standing leg gathers 32 face inward or outward. Such end strips 236 preferably comprise a fluid pervious non-woven material, but may be fluid impervious or a material other than a non-woven material. Such materials are known in the art. The end strips 236 also may help prevent the longitudinal flow of exudates past the ends of the core assembly 250, particularly if the edges of the non-woven strips overlying the core assembly 250 are left un-bonded so that they form pockets to hold exudates.

Waist elastics 5 and tummy elastics 3 may be incorporated into the garment 10 to contract the garment 10 about the wearer's abdomen. Such elastics are typically stretched as they are joined to the garment 10 so that the contraction of the elastics causes the garment 10 to contract about the wearer. The elastics also may be applied in an unstretched state and then mechanically stretched to create an elasticized region in the garment (often called a zero-strain laminate). The elastics also may be applied in an inelastic state and then heat activated to cause them to become elasticized. The elastics 3, 5, and 206 may be made from natural or synthetic rubber, elastomers, LYCRA® elastomer (available from E. I. DuPont de Nemours and Company, a business having offices in Wilmington, Del.), polyurethane, heat shrinkable polymer ribbons, or any other suitable elastic material or composite. Such materials are known in the art.

In a preferred embodiment, the waist elastics 5 are located proximal to one or both longitudinal ends 204 of the chassis layer 234, and are thereby located along the waist encircling edge of the fully assembled garment 10. In such an embodiment, the waist elastics 5 may be located on one side of the chassis layer 234, within a fold in the chassis layer 234 (as shown in FIG. 2), or otherwise fixed in the proximity of the longitudinal ends 204. U.S. Pat. No. 4,515,595 issued to Kievit et. al. and U.S. Pat. No. 4,816,025 issued to Foreman illustrate other embodiments of elasticized waist features of absorbent garments, and are hereby incorporated by reference in their entirety.

Tummy elastics 3 also may be disposed in the garment 10 between the longitudinal ends 204 and the leg opening cutouts 22 to thereby be positioned across the wearer's stomach. The tummy elastics 3 may be attached directly to the chassis layer 234 or may be disposed between a pair of carrier layers 232, 232′ to form tummy elastic assemblies 252 that are attached to the chassis layer 234. The tummy elastics 3 may be located on the interior or exterior side of the chassis layer 234, and may be covered by additional layers of material. In a preferred embodiment, the tummy elastics 3 are affixed between a pair of carrier layers 232, 232′. The carrier layers 232, 232′ preferably comprise non-woven materials, but may be made of any suitable material, and may be liquid pervious or liquid impervious. The carrier layers 232, 232′ are preferably gas pervious to allow the garment 10 to “breathe.”

In one embodiment, the tummy elastics 3 may extend across the entire width of the garment 10. In a preferred embodiment, shown in FIGS. 1 and 2, the tummy elastics 3 extend across the lateral sides of the garment 10, but not across the portion of the garment 10 overlying the absorbent core 16. Such a preferred embodiment may provide improved fit and comfort and improve the garment's appearance. U.S. Pat. No. 5,449,353 issued to Watanabe et. al. and U.S. Pat. No. 5,749,865 issued to Yamamoto et al. illustrate other embodiments of elasticized waist features of absorbent garments, and are incorporated herein by reference in their entirety.

The elastics 3, 5, 206 or any other elastics may be joined to the garment 10 by the use of a flexible adhesive or other suitable joining method. Suitable adhesives include HL-1258 by H. B. Fuller Company of St. Paul, Minn.; Findley 2031 and H2587-01 by Ato Findley Inc. of Wauwatosa, Wis.; and DISPOMELT® 34-5665 by National Starch Co. of Bridgewater, N.J. Adhesives that may be used to secure elastic elements to the absorbent garment include DISPOMELT® 34-578A by National Starch Co. of Bridgewater, N.J. In a preferred embodiment of the invention, the adhesive utilized includes HL 1486UZP, which is available from H. B. Fuller Company of St. Paul, Minn. This and other methods for attaching elastics to absorbent garments are known in the art.

As noted previously, it often is desirable for an absorbent garment to contract around various parts of the wearer's body to provide improved comfort and exudate containment. In a preferred embodiment of the present invention depicted in FIG. 2, the garment 10 further comprises at least one standing leg gather 32, and preferably at least 2 standing leg gathers 32, for improving the ability of the garment 10 to contain body exudates. The standing leg gathers 32 may be formed by incorporating a plurality of gather elastics 206 into folds in the top sheet 14 (not shown), or preferably may be provided as separate standing leg gather assemblies that are attached to the garment 10 near the leg hole cutouts 22 as shown in FIG. 2. The gather elastics 206 cause the standing leg gathers 32 to rise above the interior surface of the garment 10, thereby forming vertical curtains of material that help contain exudates. The standing leg gathers 32 may be liquid pervious or liquid impervious, and more than one pair of opposing standing leg gathers 32 may be provided.

Additional elastics (not shown) also may be incorporated into the chassis layer 234, top sheet 14 or back sheet 12 adjacent the leg hole cutouts 22 to form non-standing leg gathers 36 (FIG. 1), as is known in the art. Non-standing leg gathers 36 contract the garment 10 around the wearer's legs and body to prevent leakage. U.S. Pat. Nos. 3,860,003 and 4,081,301 issued to Buell, U.S. Pat. No. 4,695,278 issued to Lawson, U.S. Pat. No. 4,808,177 issued to Des Marais, U.S. Pat. No. 4,795,454 issued to Dragoo, and U.S. Pat. No. 4,938,755 issued to Foreman illustrate other embodiments of leg cuffs and gathers in absorbent garments, and the disclosures of these patents are hereby incorporated by reference in their entirety.

The absorbent garment 10 of the invention also preferably includes a fastening mechanism by which the front waist region 242 is associated with rear waist region 244 to form a waist hole 30. The fastening mechanism may comprise a permanent seal whereby the respective side edges 148 are attached to one another to form side seals 48 (FIG. 1). The absorbent garment 10 then can be pulled on and off like an undergarment or pair of pants. Any mechanism can be used to form the permanent seal 48. The use of the expression “permanent seal” in this context is not meant to encompass seals that cannot be broken, but rather permanent is meant to encompass sealing mechanisms that are not intended to be broken during normal use and application. The respective side edges 148 can be associated with one another to form seal 48 using techniques known in the art, including, for example, using adhesives such as hot melt adhesives and construction adhesives, chemical or solvent bonding, ultrasonic welding, stitching, heat bonding, or any other method of affixation known or hereafter discovered.

The absorbent garment 10 of the invention may also include a releasable fastening mechanism by which the front waist region 242 is associated with rear waist region 244 to form a waist hole 30. The releasable fastening mechanism may comprise a mechanism whereby the respective side edges 148 are releasably attached to one another to form side seals 48 (FIG. 1). Releasable fastening mechanisms are well known in the art, and may include tabs laterally extending from the laterally opposing rear side edges 148, adhesive strips, belts, and the like. The particular mechanism by which the respective parts of garment 10 may be adhered to one another include hook and loop type fasteners, pressure sensitive adhesives, snaps, clips, pins, and the like. Those skilled in the art are capable of manufacturing garment 10 to have either permanent or releasable fastening mechanisms, using the guidelines provided herein.

The wetness/dryness indicators 18 of the present invention preferably are in fluid communication with the absorbent core 16, and yet are visible through back sheet material 12. These wetness/dryness indicators 18 preferably are positive disappearing graphics and negative appearing graphics. One manner of arranging the respective indicators 18 is to imprint them on a separate web or multiple webs of material 180, which can be any type of material (e.g. polyolefin film, nonwoven, tissue and the like.) A particularly preferred embodiment of the invention includes imprinting the positive disappearing graphic on one surface of the web 180, and imprinting the negative appearing graphic on the opposing surface of the web 180. An alternative embodiment of the invention includes imprinting the positive disappearing graphic and the negative appearing graphic on the same surface of the web 180. In both embodiments, only one layer of web material 180 is needed, and the respective graphics may be superimposed over one another such that the negative appearing graphic appears directly over the positive disappearing graphic when the garment is wetted with a normal insult amount of urine or other bodily exudate. An alternative embodiment of the invention includes imprinting one graphic on the absorbent core facing surface of the back sheet 12, and the other graphic on the back sheet facing surface of the absorbent core 16. Other embodiments and arrangements are within the scope of the invention.

When the positive disappearing graphic and the negative appearing graphic are imprinted on a separate web(s) 180, the web 180 can be disposed on the garment 10 in any suitable manner. For example, the web 180 can be disposed on the garment 10 in its respective position using adhesives, ultrasonic or thermal bonding, or it may be friction fitted into place. It is preferred that web 180 be placed on garment 10 using cut-and-place techniques that are well known in the art, and then registering the web 180 by placing it on a specific portion of garment 10. It is known to effect synchronous, in-line placement of absorbent core pads on a continuous web of material, as described in U.S. Pat. No. 5,415,716, the disclosure of which is incorporated by reference herein in its entirety. It further is known that graphics can be applied in registration on a moving sheet of material that ultimately is used in an absorbent garment by controlling the timing of placing the discrete graphic material. A number of documents describe placing graphic materials in registration on a moving web, including U.S. Pat. Nos. 6,165,306, 6,149,755, 6,095,218, 6,074,333, and 6,059,710, the disclosures of each of which are incorporated by reference herein in their entireties. Any of the methods and apparatus described in these documents can be used in the invention to place the web 180 on the absorbent garment 10.

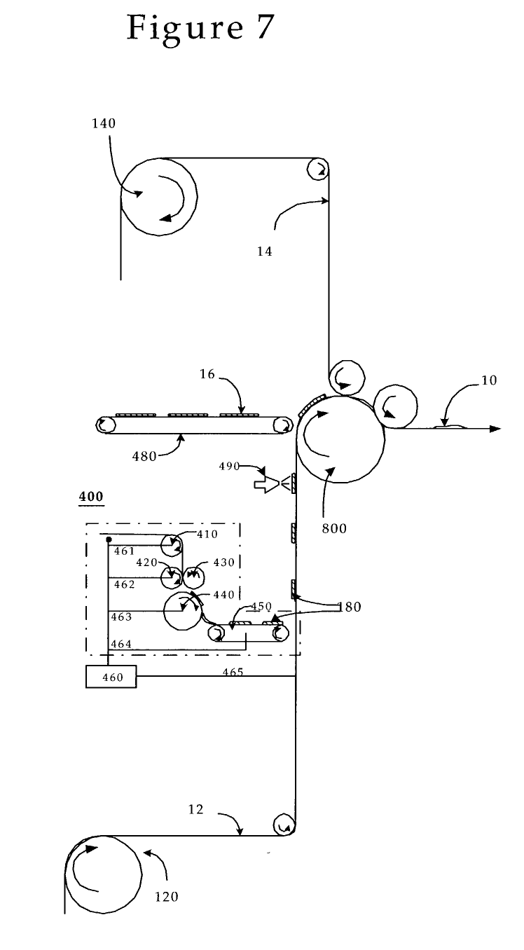

The methods and apparatus suitable for placing web 180 of the invention will be explained in further detail with reference to FIG. 7. Any cut-and-place apparatus 400 capable of cutting a material from a moving web at a first speed, and placing it on another moving web at a second speed different from the first speed can be used in the present invention.

Suitable cut-and-place apparatus are disclosed in, inter alia, U.S. Pat. Nos. 6,165,306, 6,149,755, 6,095,218, 6,074,333, 6,059,710, and 5,415,716, the disclosures of each of which are incorporated by reference herein in their entireties. The simplified cut-and-place apparatus illustrated in FIG. 7 includes a feed roller system 410 that feeds a web of material 180 containing the graphic at a first speed to the cut-and-place apparatus 400. Those skilled in the art will recognize that feed roller system 410 may be comprised of any number of component parts, such as a supply reel, a plurality of dancer rolls and edge guide rolls, and pull rolls to establish the first speed of the web material.

Feed roller system 410 feeds the web of material to a cutting station, illustrated in FIG. 7 as a pair of rollers in cutting engagement with one another. Any type of cutting device can be used in the present invention that is capable of controlled cutting of a moving web of material. FIG. 7 shows a rotatable knife roll 420 in cutting engagement with a rotatable anvil roll 430. The knife roll can be controlled to control cutting the web at the appropriate locations to form an appropriately sized web 180.

Upon cutting, the web 180 is placed on a rotatable vacuum transfer drum 440 that alters the velocity of the moving web to the second speed so as to control its placement on, for example, an inner surface of back sheet 12, which is moving at a speed different from the speed at which the web 180 containing the graphics is fed to cut-and-place apparatus 400. FIG. 7 illustrates an embodiment where the web 180 first is transferred to back sheet 12, and then placed in registration on garment 10 at placement forming station 800. Moving web 12 also may be chassis layer(s) 234, as will be appreciated by those skilled in the art.

The rotatable vacuum transfer drum 440 can be any type of rotating drum capable of drawing a vacuum so that it can grab onto and hold the now severed web 180, and ultimately transfer it to garment 10 via conveyor 450, and back sheet 12, for example. In this embodiment, conveyor 450 transports the web 180 at the second speed to the moving back sheet 12, and preferably, the speed of the web that contains garment 10. Skilled artisans will appreciate, however, that rotatable vacuum transfer drum 440 could deposit the web 180 on a suitable surface of back sheet 12 or absorbent core 16, without the use of the conveyor 450.

The cut-and-place apparatus 400 can be controlled by controller 460. Controller 460 monitors and controls the first speed at which the moving web is conveyed by monitoring and controlling feed roller system 410 via control 461, and by monitoring and controlling knife roll 420 and anvil roll 430 via control 462. Controller 460 also monitors and controls the second speed at which the severed web 180 is conveyed by monitoring and controlling the rotatable vacuum transfer drum 440 via control 463, the conveyor 450 via control 464, and by monitoring the speed of moving back sheet 12 via control 465. Those skilled in the art will recognize that various modifications may be made to controller 460 to adequately control the first and second speeds such that the web 180 is accurately placed on an appropriate surface of back sheet 12, absorbent core 16, or other element of garment 10. Using the guidelines provided herein, those skilled in the art are capable of designing a suitable controller 460 without undue experimentation.

FIG. 7 illustrates the web 180 containing the wetness/dryness indicators 18 being disposed on an interior surface of the back sheet 12, but the invention is by no means limited to this particularly preferred embodiment and configuration. The web 180 could be disposed on a back sheet facing surface of absorbent core 16. In addition, two or more webs 180 could be disposed on either of the aforementioned surfaces of the back sheet 12 and absorbent core 16, using additional cut-and-place apparatus.

The embodiment shown in FIG. 7 illustrates an adhesive applicator 490 applying adhesive to web 180 to ensure its attachment to garment 10. Application of adhesive via applicator 490 is optional in the present invention. Adhesive could be applied to either or both, or neither surface of web (i.e., absorbent core facing surface and back sheet facing surface).

Any mechanism 490 capable of supplying an adhesive, albeit a spray adhesive, or one that is “rubbed” on, can be used in the invention, such as an extrusion applicator, a stencil applicator, or a printing applicator. Suitable adhesives include any adhesive commonly employed in absorbent garments that is useful in adhering one or more components to together. It is particularly preferred to use construction adhesives, including HL-1258 by H. B. Fuller Company of St. Paul, Minn.; Findley 2031 and H2587-01 by Ato Findley Inc. of Wauwatosa, Wis.; and DISPOMELT® 34-5665 by National Starch Co. of Bridgewater, N.J. Other adhesives that may be used in the invention include DISPOMELT® 34-578A, available from National Starch Co. of Bridgewater, N.J. Any of these adhesives may be used in all adhesive applications in the absorbent garment, or only in select applications as a construction adhesive for bonding parts of the garment as the top sheet, back sheet, absorbent core, and additional layer(s).

It is preferred in the invention that garment 10 be formed at garment forming station 800 where web 180 is disposed at least partially between the absorbent core 16 and the back sheet 12. It will be appreciated, however, that web 180 could be disposed on the back sheet facing surface of absorbent core 16, or on an additional layer(s) (not shown) and then secured to the remaining elements of garment 10 at garment forming station 800, using the techniques described herein.

The absorbent core 16 of the invention preferably is used immediately after it is formed as a component part of the absorbent garment 10. In this context, the absorbent cores 16 would be transported to garment forming station 800 via core conveyor 480 (or any other conveying device) where they will be disposed at least partially between a top sheet 14 and a back sheet 12. Top sheet material 14 may be supplied to forming station 800 by top sheet supply mechanism 140, which can be any supply mechanism capable of supplying top sheet 14 to garment forming station 800. Preferably, top sheet material 14 is supplied via a supply roller 140 and select feed or guide rollers. Back sheet material 12 likewise can be supplied to forming station 800 by back sheet supply mechanism 120, which can be any supply mechanism capable of supplying back sheet 12 to garment forming station 800. Preferably, back sheet material 12 is supplied via a supply roller 120 and select feed or guide rollers. Forming station 800 brings together the respective components of absorbent article 10 by disposing absorbent core 16 between top sheet material 14, and back sheet material 12. After placing disposal fastening mechanism on garment 10, the final absorbent garment 10 then may be cut and folded to the appropriate size and shape downstream from forming station 800.

The embodiment illustrated in FIG. 7 could include a number of additional devices used in preparing absorbent garments. For example, it is conventional to employ leg elastics 36, and standing leg gathers 32 (FIGS. 1 and 2). Leg elastics 36 could be supplied to garment forming station 800 by any suitable manner known in the art. It is conventional to apply adhesives to either the leg elastics 36 themselves, or to apply adhesives on back sheet 12 or top sheet 14, and then dispose the leg elastics 36 there between. The leg elastics 36 may be supplied as an elastic element, or as a laminate of elastic elements disposed between two outer layers, preferably outer non-woven materials.

Standing leg gathers 32 can be applied as a separate material to top sheet 14, and disposed on the exterior facing side (e.g., body facing side) of the top sheet 14. The standing leg gathers 32 can be applied either prior to, or downstream from, garment forming station 800 using techniques known in the art. The standing leg gathers 32 may be secured in place on garment 10 by placing outer sheets, preferably, outer non-woven sheets 236 over the longitudinal end portions of the standing leg gathers 32.

Other fastening elements also can be supplied and attached to garment 10 either prior to or downstream from garment forming station 800. It is preferred that fastening elements are secured to garment 10 after garment forming station 800. Additional layers 20 (FIG. 2) may be placed on or in garment 10 before or after forming station 800, or be disposed between top sheet 14 and back sheet 12 by supplying the additional layer(s) to garment forming station 800. These additional layer(s) 20 also may be cut-and-placed on top sheet 14 and/or back sheet 12 using a cut-and-place apparatus 400.

It also is typical in the industry to include a waist elastic system comprising one or more waist elastic materials 5. Waist elastics 5 preferably are supplied upstream of garment forming station 800, and thus disposed between the top sheet 14 and back sheet 12. Waist elastics 5 may, however, be supplied downstream from garment forming station 800, and placed within a fold at longitudinal ends 204 of an outer cover or chassis layer(s) 234 to be disposed outside the back sheet 12, using techniques known in the art. The waist elastic elements 5 may be supplied as a layer of material, or as elastic elements disposed between two outer materials. In a similar fashion, tummy elastics 3 may be supplied prior to, or downstream from garment forming station 800.

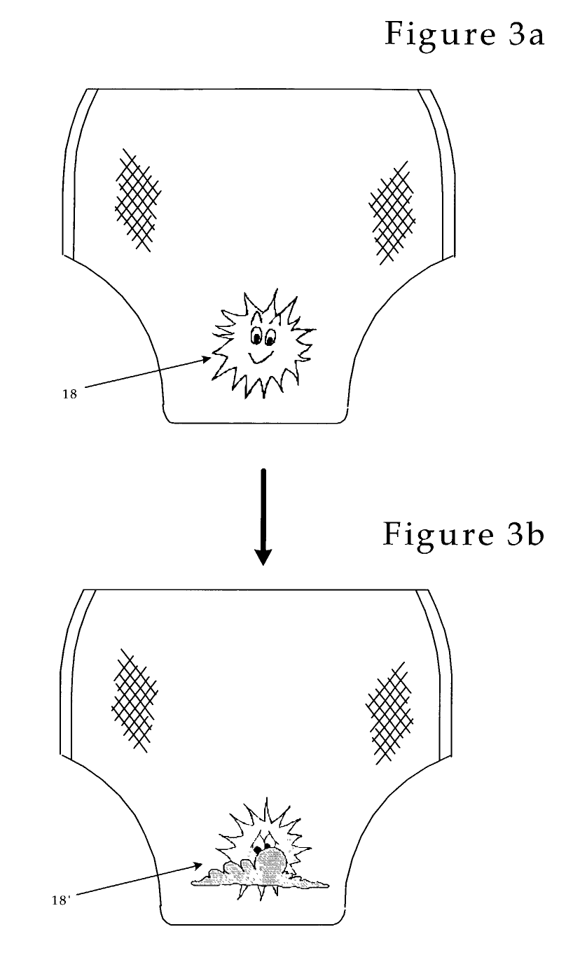

Turning now to FIGS. 3-6, various embodiments of the invention will be described with reference to particularly preferred embodiments. FIG. 3a depicts a training pant with a positive disappearing graphic 18 in the crotch or insult region of the garment. The positive disappearing graphic 18 is visible during normal use, and prior to wetting. Here, the positive disappearing graphic 18 is a smiling sun. FIG. 3b depicts the same training pant as in FIG. 3a, only now the training pant has been wetted with a normal insult amount of urine. A negative appearing graphic 18′ now has appeared in or around the same region as the positive disappearing graphic 18. Here, the negative appearing graphic 18′ is a frowning sun behind a rain cloud. The negative appearing graphic 18′ need not appear in the same area as the positive disappearing graphic 18 as shown in FIGS. 3-6, but rather may appear in a different area. It is preferred, however, that the respective graphic elements appear and disappear in the same general area to assist in potty training the garment wearer.

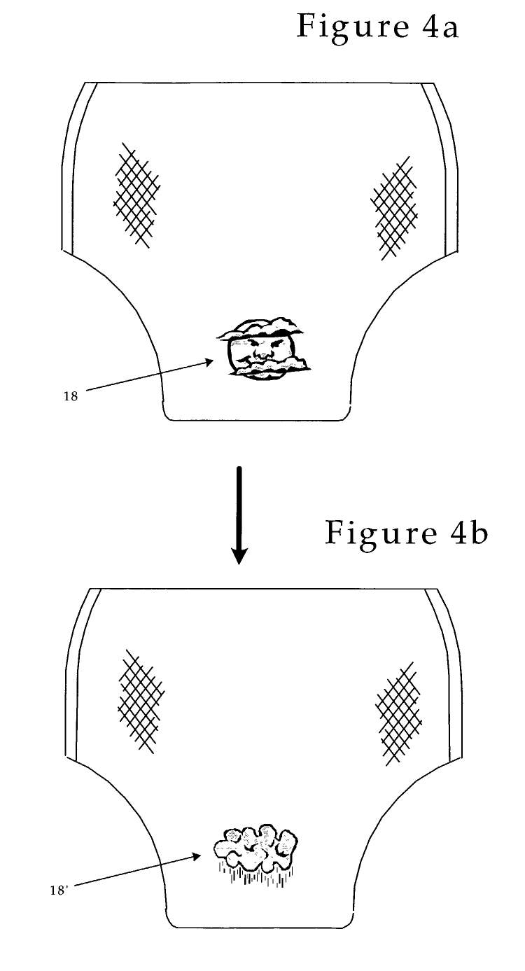

Another embodiment of the invention is shown in FIGS. 4a and 4b. FIG. 4a illustrates a training pant with a positive disappearing graphic 18, where the graphic is a smiling sun behind a few friendly clouds. Upon wetting, the smiling sun behind a few friendly clouds disappears, and a dark rain cloud appears as the negative appearing graphic 18′, thereby indicating to the wearer, and to the caregiver that the absorbent article has been wetted. Again, appearing graphic 18′ need not appear in the exact same position as disappearing graphic 18, nor do the graphics need to be related to one another. For example, the invention is not limited to graphics 18, 18′ that are related in any way, such as a shining sun that turns into a rain cloud. Rather, the graphics 18, 18′ could be completely unrelated to one another, such as a smiling teddy bear as the positive disappearing graphic 18, and a lightning bolt as the negative appearing graphic 18′.

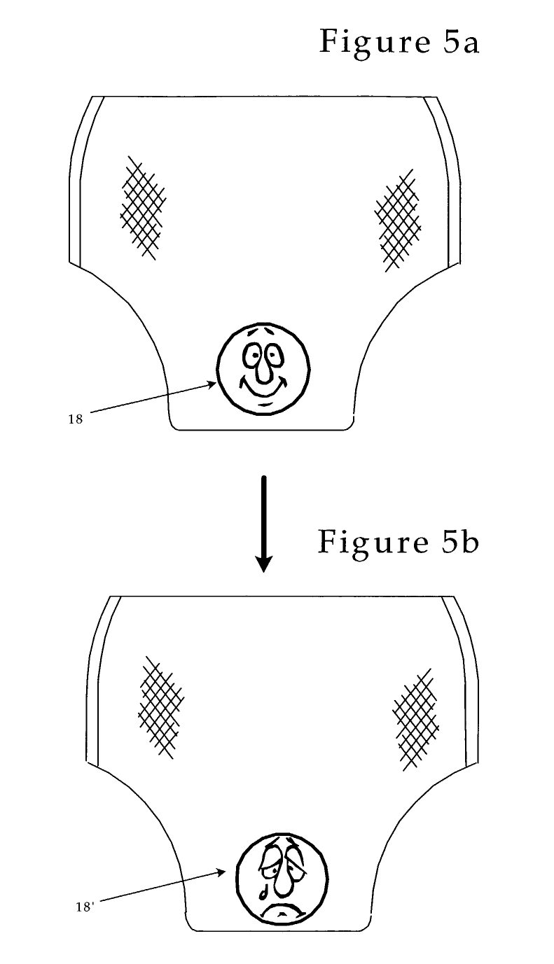

Another embodiment is illustrated in FIGS. 5a and 5b. FIG. 5a shows an absorbent garment with a smiling cartoon as the positive disappearing graphic 18, and FIG. 5b depicts an absorbent garment with a sad cartoon face as the negative appearing graphic 18′. The cartoon characters, just like any other graphics useful in the absorbent garment of the present invention, may be in black-and-white, or may be colored.

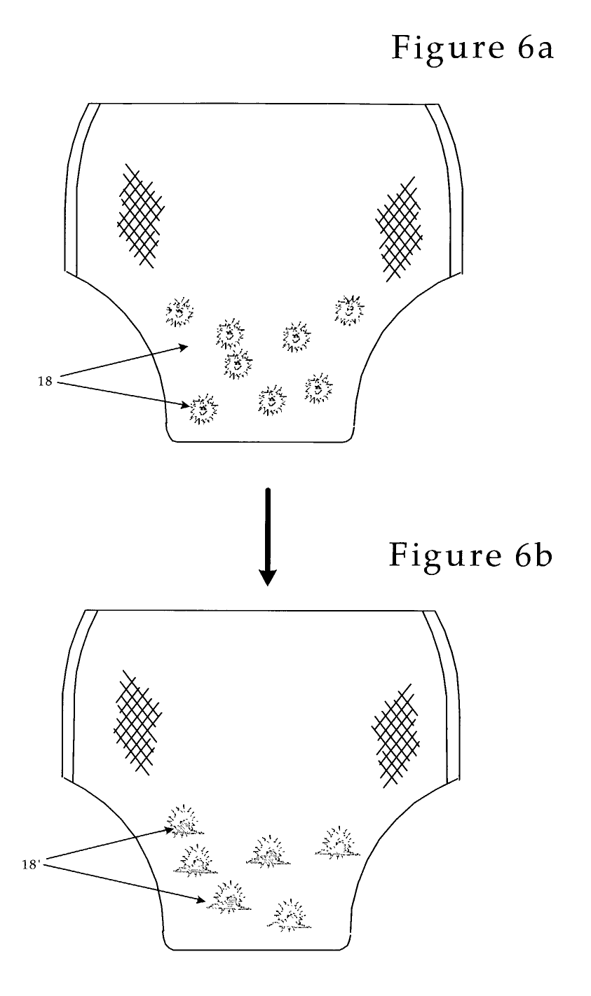

FIGS. 6a and 6b illustrate yet another preferred embodiment of the present invention. FIG. 6a depicts an absorbent garment that includes a plurality of positive disappearing graphics 18. When wetted, the garment of FIG. 6a is transformed into the garment shown in FIG. 6b, which includes a plurality of negative appearing graphics 18′. The plurality of graphic elements may be disposed throughout the garment, and the negative appearing graphics 18′ need not necessarily appear in the same places as the positive disappearing graphics 18. In addition, the respective graphic elements need not be of the same size or shape, and need not be related to one another.

Use of positive disappearing graphics 18 and negative appearing graphics 18′ will aid the caregiver in training the wearer to use the toilet. It is well established that individuals learn better through positive reinforcement. Thus, the wearer is met with positive images each time he or she uses the toilet, and does not insult the absorbent garment. That is, the positive disappearing graphic 18 remains visible to the wearer. In a similar manner, the negative appearing graphics 18′ inform the wearer that an accident has happened without having to resort other forms of punishment that which might otherwise discourage the wearer from potty training. The combination of the positive disappearing graphic 18, and the negative appearing graphic 18′ therefore provide an excellent potty training aid.

The garments depicted in FIGS. 3a-6b reveal only the positive disappearing graphics 18 and the negative disappearing graphics 18′. Those skilled in the art will appreciate, however, that the absorbent garments of the present invention can include any number of additional graphic elements, each of which may or may not bear some relationship to the wetness/dryness indicators. Any of the graphic elements disclosed in U.S. Pat. Nos. 6,297,424 and 6,307,119 may be used in the present invention. To the extent that the positive disappearing graphics 18 and negative appearing graphics 18′ are related in some manner to other graphical elements, this arrangement may aid the caregiver in telling a story to the wearer to encourage potty training. The respective graphic elements may be registered with respect to one another as disclosed, for example, in U.S. Pat. No. 5,766,389, the disclosure of which is incorporated by reference herein in its entirety.

The positive disappearing graphics 18 can be printed on any of the elements of the absorbent garment 10 using techniques known in the art. The graphics 18 can be printed directly on the back sheet 12, the absorbent core 16, or an intermediate web 180. The disappearing graphics preferably are printed with disappearing ink that disappears when wetted, or when heated. The amount of fluid and/or heat needed to make the ink disappear should be less than the amount of fluid and/or heat generated during a normal insult of urine, which will depend upon the size of the absorbent garment (and likewise the size of the wearer). Those skilled in the art are capable of using a suitable ink for any of the graphic elements described herein, using the guidelines provided in this disclosure.

Suitable inks or dyes useful in printing the positive disappearing graphics 18 of the present invention are those that disappear when subjected to liquid and/or heat. Preferably, the ink(s) used to print positive disappearing graphic 18 is a water soluble ink of the type disclosed in U.S. Pat. No. 4,022,211. Examples of water soluble ink formulations include a water-soluble polyvinyl alcohol diluted with water, combined with appropriate coloring agents. For instance, a blue water soluble ink could be formulated from a 50% solution of Cascorex EA 9065, a polyvinyl alcohol from Borden Adhesives, diluted with distilled water with the addition of 0.1% by weight of GAF Neptune Blue BRA dye. Another suitable formulation is a solution comprised of 50% water, 50% of water-soluble polyvinyl alcohol (Cascorex EA 9065, about 8% solids from Borden Chemical Company), colored with a tissue dye (Sky Blue 6BX from E. I. DuPont Company) in the amount of 0.5% by weight. Coloring agents used in these formulations could be substituted with others, to produce different colors. Examples of other suitable coloring agents include: Pontamine Turquoise 8 GLP (a direct blue dye), Bond yellow CS (a direct yellow dye), DuPont Red 8BLX (a direct red dye), Rhodamine B Extra (a basic red dye), and Paper Blue R (a direct dye) all available from E. I. DuPont Company; and EASTACRYL dark red dye available from Eastman Kodak Company. Coloring agents also could be added in different concentrations to produce different color intensities. Those skilled in the art are capable of designing and manufacturing a suitable disappearing ink for use in the invention, using the guidelines provided herein.

The positive disappearing graphics 18 can be printed on any of the elements of the absorbent garment 10 using techniques known in the art. The negative appearing graphics 18′ can be printed directly on the back sheet 12, the absorbent core 16, or an intermediate web 180. A preferred embodiment of the invention includes the use of an intermediate web 180 with the positive disappearing graphics 18 printed on one side, and the negative appearing graphics 18′ printed on the opposing side (and printed so that it will appear in its correct position when viewed through web 180). The negative appearing graphics 18′ preferably are printed with appearing ink that appears when wetted, or when heated.

Suitable inks or dyes useful in printing the negative appearing graphics 18′ of the present invention are those that appear when subjected to liquid and/or heat. Preferably, the ink(s) used to print negative appearing graphics 18′ is of the type disclosed in U.S. Pat. Nos. 4,292,916, 4,812,053, 4,903,254, 4,987,849, 5,045,283, 5,053,339, and 5,058,088, the disclosures of each of which are incorporated by reference herein in their entirety. Examples of suitable inks include ink formulations which are sensitive to pH (i.e. the pH of urine, when in contact with the ink, causes the printed graphic to appear.) Those skilled in the art are capable of designing and manufacturing a suitable appearing ink for use in the invention, using the guidelines provided herein.

The graphics of the present invention also may be formed from appearing or disappearing adhesives that change colors or disappear or appear when wetted. The graphics also may be printed with pH sensitive inks that disappear and/or appear when the surrounding pH changes due to, for example, an insult with urine. Any other type of ink or dye that is suitable in forming a positive disappearing graphic 18 and/or a negative appearing graphic 18′ can be used in the present invention.

While the invention has been described in detail with reference to particularly preferred embodiments and examples, those skilled in the art will appreciate that various modifications may be made to the invention without departing from the spirit and scope thereof.

What is claimed is:

1. An absorbent garment comprising:
a top sheet;
a back sheet;
an absorbent core disposed at least partially between the top sheet and the back sheet; and
at least one disappearing graphic and at least one appearing graphic that disappear and appear respectively, when the absorbent article is insulted with an average insult amount of urine.

2. The absorbent article as claimed in claim 1, wherein the at least one disappearing graphic and/or the at least one appearing graphic are disposed at least partially between the back sheet and the absorbent core.

3. The absorbent article as claimed in claim 1, wherein the at least one disappearing graphic and/or the at least one appearing graphic are disposed on an absorbent core facing surface of the back sheet.

4. The absorbent article as claimed in claim 1, wherein the at least one disappearing graphic and/or the at least one appearing graphic are disposed on a back sheet facing surface of the absorbent core.

5. The absorbent article as claimed in claim 1, further comprising a web disposed at least partially between the absorbent core and the back sheet, and in fluid communication with the absorbent core, the web having printed thereon the at least one disappearing graphic and at least one appearing graphic.

6. The absorbent article as claimed in claim 5, wherein the at least one disappearing graphic and at least one appearing graphic are printed on the same surface of the web.

7. The absorbent article as claimed in claim 5, wherein the at least one disappearing graphic and at least one appearing graphic are printed on opposing surfaces of the web.

8. The absorbent article as claimed in claim 1, wherein the at least one disappearing graphic is a positive graphic, and the at least one appearing graphic is a negative graphic.

9. The absorbent article as claimed in claim 8, wherein the at least one positive disappearing graphic and/or the at least one negative appearing graphic are disposed at least partially between the back sheet and the absorbent core.

10. The absorbent article as claimed in claim 8, wherein the at least one positive disappearing graphic and/or the at least one negative appearing graphic are disposed on an absorbent core facing surface of the back sheet.

11. The absorbent article as claimed in claim 8, wherein the at least one positive disappearing graphic and/or the at least one negative appearing graphic are disposed on a back sheet facing surface of the absorbent core.

12. The absorbent article as claimed in claim 8, further comprising a web disposed at least partially between the absorbent core and the back sheet, and in fluid communication with the absorbent core, the web having printed thereon the at least one positive disappearing graphic and at least one negative appearing graphic.

13. The absorbent article as claimed in claim 8, wherein the at least one positive disappearing graphic and at least one negative appearing graphic are printed on the same surface of the web.

14. The absorbent article as claimed in claim 8, wherein the at least one positive disappearing graphic and at least one negative appearing graphic are printed on opposing surfaces of the web.

15. A method of making an absorbent article comprising:
providing a top sheet;
providing a back sheet;
providing an absorbent core and disposing the absorbent core at least partially between the top sheet and the back sheet; and
disposing at least one disappearing graphic and at least one appearing graphic at least partially between the absorbent core and the back sheet,
whereby the at least one disappearing graphic and the at least one appearing graphic disappear and appear, respectively, when the absorbent article is insulted with an average insult amount of urine.

16. The method as claimed in claim 15, wherein the at least one disappearing graphic and/or the at least one appearing graphic are disposed on an absorbent core facing surface of the back sheet.

17. The method as claimed in claim 15, wherein the at least one disappearing graphic and/or the at least one appearing graphic are disposed on a back sheet facing surface of the absorbent core.

18. The method as claimed in claim 15, further comprising providing a web having printed thereon the at least one disappearing graphic and at least one appearing graphic; and
disposing the web at least partially between the absorbent core and the back sheet such that the web is in fluid communication with the absorbent core.

19. The method as claimed in claim 15, wherein the at least one disappearing graphic and at least one appearing graphic are printed on the same surface of the web.

20. The method as claimed in claim 15, wherein the at least one disappearing graphic and at least one appearing graphic are printed on opposing surfaces of the web.

21. The method as claimed in claim 15, wherein the at least one disappearing graphic is a positive graphic, and the at least one appearing graphic is a negative graphic

22. The method as claimed in claim 21, wherein the at least one positive disappearing graphic and/or the at least one negative appearing graphic are disposed on an absorbent core facing surface of the back sheet.

23. The method as claimed in claim 21, wherein the at least one positive disappearing graphic and/or the at least one negative appearing graphic are disposed on a back sheet facing surface of the absorbent core.

24. The method as claimed in claim 21, further comprising providing a web having printed thereon the at least one positive disappearing graphic and at least one negative appearing graphic; and
disposing the web at least partially between the absorbent core and the back sheet such that the web is in fluid communication with the absorbent core.
